(19) United States
(12) Patent Application Publication
(10) Pub. No.: US 2006/0257336 A1
(43) Pub. Date: Nov. 16, 2006

(54) Cosmetic composition comprising silica particles, reflecting particles, and at least one polymer, preparative processes, and uses thereof

(76) Inventors: Veronique Ferrari, Maisons-Alfort (FR); Helene Khachikian, Alfortville (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/406,371

(22) Filed: Apr. 19, 2006

Related U.S. Application Data
(60) Provisional application No. 60/675,455, filed on Apr. 28, 2005.

(30) Foreign Application Priority Data
Apr. 19, 2004 (FR) 05 03895

(51) Int. Cl. A61K 8/29; A61K 8/88

(52) U.S. Cl. 424/59; 424/401; 424/70.17

(57) ABSTRACT

Disclosed herein is a cosmetic skin make-up and/or care composition comprising an oily phase, for example, a gelled or structured oily phase, comprising silica particles and reflecting particles, said oily phase comprising at least one polymer having a weight-average molecular weight of less than 100,000, for example, below 50,000, comprising (a) a polymer skeleton comprising hydrocarbon repeat units including at least one heteroatom, and (b) optionally at least one pendant fatty chain and/or at least one terminal fatty chain which may be optionally functionalized, comprise from 6 to 120 carbon atoms and which are bonded to the hydrocarbon repeat units. Also disclosed herein is a process for the preparation of this cosmetic composition, to a gloss comprising this cosmetic composition and to a method for obtaining a glossy deposit comprising applying this cosmetic composition to a substrate.

This application claims benefit of U.S. Provisional Application No. 60/675,455, filed Apr. 28, 2005, the contents of which are incorporated herein by reference. This application also claims benefit of priority under 35 U.S.C. § 119 to French Patent Application No. FR 0503895, filed Apr. 19, 2005, the contents of which are also incorporated herein by reference.

At least one aspect of the present disclosure relates to the field of skin make-up and/or care, for example, the field of skin make-up and/or care compositions comprising an oily phase containing silica particles.

Another aspect of the present disclosure relates to compositions in which the silica particles are used, for example, as a main gelling system, but the presence of an additional gelling agent in these compositions is not excluded.

Skin make-up and/or care compositions comprising a gelled or structured oily phase, such as make-up foundations, rouges, eye shadows, and lipsticks, are commonly used to change the appearance of the face, for example, to enhance the cheekbones or make the lips pulpous.

These skin make-up and/or care compositions are generally sensitive to shear, especially during their manufacture, and, when these compositions comprise suspended particles, the sensitivity to shear means that these particles tend to settle during storage.

These compositions comprising a gelled or structured oily phase can be used, for example, in lip make-up compositions of the gloss type, since the gelling of the phase produces opalescent systems. This transparency is of additional interest if the refractive indices of the oils of the oily phase have been chosen so as to allow a glossy deposit on the lips.

It is therefore useful to provide skin make-up and/or care compositions comprising an oily phase, for example, an oily phase gelled or structured by means of silica particles, these compositions having improved homogeneity and improved stability. For example, when these compositions comprise suspended particles, it would be useful to improve the stability of the particle suspension, i.e., to provide a composition in which the particles do not tend to settle out.

The present inventors have discovered that the problem with the stability of these compositions can be solved by using a specific polymer, for example, a polymer having a weight-average molecular weight of less than 100,000, for instance, less than 50,000, comprising (a) a polymer skeleton comprising hydrocarbon repeat units including at least one heteroatom, and (b) optionally at least one pendant fatty chain and/or at least one terminal fatty chain which may be optionally functionalized, comprise from 6 to 120 carbon atoms, and which are bonded to the hydrocarbon repeat units.

Disclosed herein is a cosmetic skin make-up and/or care composition comprising an oily phase comprising suspended silica particles and reflecting particles and at least one polymer having a weight-average molecular weight of less than 100,000, for example, less than 50,000, this polymer comprising (a) a polymer skeleton comprising hydrocarbon repeat units including at least one heteroatom, and (b) optionally at least one pendant fatty chain and/or at least one terminal fatty chain which may be optionally functionalized, may comprise from 6 to 120 carbon atoms, and which are bonded to the hydrocarbon repeat units.

Also disclosed herein is a process for the preparation of this cosmetic composition which comprises mixing silica particles, reflecting particles, and at least one polymer having a weight-average molecular weight of less than 100,000, comprising (a) a polymer skeleton comprising hydrocarbon repeat units including at least one heteroatom, and (b) optionally at least one pendant fatty chain and/or at least one terminal fatty chain which may be optionally functionalized, comprise from 6 to 120 carbon atoms, and which are bonded to the hydrocarbon repeat units.

The present disclosure may make it possible to obtain transparent compositions. This property is of greatest interest when said composition is used to manufacture a gloss, because in this case the gloss properties of the materials used, such as the oils of the oily phase or the reflecting particles, may be preserved or even improved.

Thus, further disclosed herein is a gloss comprising a cosmetic composition in accordance with the present disclosure.

Also disclosed herein is a method for obtaining a glossy deposit comprising applying a cosmetic composition in accordance with the present disclosure to a substrate.

Other characteristics, features, subjects, and advantages of the present invention will become even more clearly apparent upon reading the description and examples which follow.

Oily Phase

In at least one embodiment, the oily phase of the composition according to the present disclosure may be gelled or structured.

As used herein, the term “gelled oily phase” is understood to mean that said phase is in the form of a gel, i.e., a three-dimensional network of molecules that retains a substantial amount of solvent in its meshes. The formation of such a network constitutes the gelling of said phase.

As used herein, the term “structured oily phase” is understood to mean that said phase is a rigid gel in the form of a cup or a stick.

In at least one embodiment, the gelled or structured phase used in the composition according to the present disclosure may have a dynamic viscosity at room temperature ranging from 30 to 60 Pa·s, for example, ranging from 40 to 60 Pa·s.

The dynamic viscosity of the composition may be measured with a METTLER RM 180 viscometer. The METTLER RM 180 apparatus (Rhéomat) can be equipped with different spindles according to the order of magnitude of the viscosity to be measured. For a viscosity ranging from 8 to 122 Pa·s, the apparatus may be equipped with a no. 5 spindle. The speed of rotation of the spindle is 200 rpm.

In at least one embodiment, the compositions according to the present disclosure may be transparent, this transparency being evaluated visually on the basis of a 10 μm layer thickness. This thickness corresponds approximately to the thickness of a deposit of make-up obtained, e.g., with a make-up foundation or a lipstick, for example, of the gloss type.

As used herein, the term “gloss” denotes a product that is intended to be applied to the lips and may be packaged, e.g., in a receptacle provided with an applicator, this applicator having a prehensile element that also serves as a cap for closing the receptacle.

The compositions according to the present invention may further comprise a physiologically acceptable medium, i.e., a non-toxic medium that may be applied to the skin, lips, and/or superficial body growths of a human being.

In one embodiment of the present disclosure, the cosmetic composition may comprise an oily phase with a refractive index ranging from 1.47 to 1.51, which may afford a relatively high gloss. Said oily phase of the composition according to the present disclosure may comprise silica particles; for example, the oily phase may be gelled or structured by means of silica particles.

Silica Particles

The silica particles may be chosen from pyrogenic silicas which have optionally been hydrophobized on the surface, having particle sizes of less than 1 μm. It is also possible to chemically modify the surface of the silica by means of a chemical reaction that reduces the number of silanol groups present on the surface of the silica. For example, it is possible to replace silanol groups with hydrophobic groups to give a hydrophobic silica. The hydrophobic groups may be chosen, for example, from:

trimethylsiloxy groups, which may be obtained by treating pyrogenic silica in the presence of hexamethyldisilazane. Silicas treated in this way are called “silica silylates” according to CTFA (6th edition, 1995). They are marketed, for example, under the references Aerosil R812® by DEGUSSA and CAB-O-SIL TS-530® by CABOT; or

dimethylsiloxy and polydimethylsiloxane groups, which may be obtained by treating pyrogenic silica in the presence of polydimethylsiloxane or dimethyidichlorosilane. Silicas treated in this way are called “silica dimethyl silylates” according to CTFA (6th edition, 1995). They are marketed, for example, under the references Aerosil R972® and Aerosil R974® by DEGUSSA and CAB-O-SIL TS-610® and CAB-O-SIL TS-720® by CABOT.

The hydrophobic pyrogenic silica may have a particle size ranging from nanometric to micrometric, for example, ranging from 5 to 200 nm.

The silica particles may be present in the composition in an amount ranging from 0.1% to 12%, for example, from 0.5% to 10%, or from 6% to 8% by weight, relative to the total weight of the composition.

Reflecting Particles

The reflecting particles used should be compatible with use in cosmetics and should be able to subsist in the physiologically acceptable medium; for example, they should not dissolve, or in any case, should not dissolve completely, in said medium.

The reflecting particles may be chosen from particles having a natural or synthetic substrate that is at least partially coated with at least one layer of at least one metal, particles having a synthetic substrate that is at least partially coated with at least one layer of at least one metal compound, for example, a metal oxide, particles formed of a stack of at least two layers with different refractive indices, for example, two layers of polymers, and particles of metal oxides.

The reflecting particles may be homogeneously dispersed in the composition, for example, in an amount ranging from 0.1% to 20% by weight, for instance, from 1% to 15% by weight, or from 1% to 10% by weight, e.g., 2%, relative to the total weight of the composition.

The proportion of reflecting particles may depend, inter alia, on the nature of the substrate that is intended to receive the cosmetic composition, as well as on the nature of the physiologically acceptable medium, and the nature and size of the reflecting particles. The proportion of reflecting particles may be chosen in such a way that the excessively glossy spots are distributed discretely over the surface to be made up and/or nurtured. The reflecting particles may be present in a sufficient amount for it to be possible, when the cosmetic composition is applied to a substrate such as the lips, to simultaneously observe a plurality of excessively glossy spots, for example, more than ten, or more than fifty, or even more, for instance, more than a hundred, or more than several hundred.

The size of the reflecting particles may be compatible with the manifestation of a specular reflection of visible light (400-700 nm) that is of sufficient intensity to create an excessively glossy spot, taking into account the average gloss of the composition. This particle size is capable of varying with the chemical nature of the particles, their shape, and their capability in terms of the specular reflection of visible light.

Some of the reflecting particles which can be used in the invention may exhibit a relative shift Δ, defined by the formula Δ=[L*_{SCI}−L*_{SCE}]/L*_{SCE}, greater than or equal to 0.25. By comparison, some pearlescent products that are not suitable as reflecting particles have a coefficient Δ below 0.25. In the above formula, L*_{SCI} denotes the clarity L* measured using a MINOLTA CM-2002 spectrocolorimeter in a mode called “specular component included”, and L*_{SCE} denotes the clarity L* measured using the same apparatus in a mode called “specular component excluded”. To make the measurements, a 5% by weight dispersion of the test particles is prepared in a transparent nail varnish of conventional composition (essentially nitrocellulose, a resin, and a plasticizer) and a 300 μm thick layer of the composition formed is spread in the fluid state over the black background of a contrast card.

The SCI/SCE function of the spectrocolorimeter is used with the geometry d/8 to measure L*_{SCI} and L*_{SCE}.

By way of example, the relative shift Δ measured on REFLECKS® reflecting particles marketed by ENGELHARD, containing a glass substrate coated with brown iron oxide, was more than 0.7, whereas the relative shift measured on FLAMENCO® pearlescent products marketed by the same company was below 0.2.

In at least one embodiment, the reflecting particles may have a dimension of at least 10 μm, for example, ranging from 20 μm to 80 μm.

As used herein, the term “dimension” denotes the dimension of half the population according to the statistical particle size distribution and is called D50. The size of the reflecting particles may depend on their surface state. The dimension decreases a priori as the reflectance increases, and vice versa.

From an aesthetic point of view, except where they shine to create excessively glossy spots, the reflecting particles may not be perceptible at all or not easily perceptible to the naked eye on the surface of the composition applied to its substrate. It is also desirable for the reflecting particles not to have dimensions such that they create an uncomfortable sensation on the substrate. Thus, in one embodiment, the reflecting particles may have a size less than or equal to 250 μm, for example, less than or equal to 150 μm, or less than or equal to 100 μm. The particle size may also depend on the nature of the substrate to which the composition is intended to be applied; for example, some parts of the body or face may tolerate larger dimensions more easily than other parts, without generating discomfort.

The reflecting particles may have a variety of shapes. For example, they may be in the shape of wafers or pellets, such as spherical pellets.

As used herein, the expression “in the shape of wafers” denotes particles for which the ratio of the largest dimension to the thickness is greater than or equal to 5, for example, greater than or equal to 10, or greater than or equal to 20. The thickness of the particles in the shape of wafers may range, for example, from 0.5 μm to 5 μm.

Particles having a substantially planar external surface are suitable for use in one embodiment of the present invention because they can more easily give rise to an intense specular reflection if their size, structure, and surface state allow it. This effect is referred to as a “mirror effect.”

For such particles, the light that reflects in a direction that forms, together with the normal to the reflecting surface, the same angle as that formed by the incident light with this normal, enables these particles to appear as excessively glossy spots, and not the light diffused in other directions.

In one embodiment, it may be desirable for the reflecting particles to be non-diffusing and non-matt.

In another embodiment, it may also be desirable for the reflecting particles not to substantially change the coloration of the cosmetic composition.

In this embodiment, reflecting particles which allow a metallic reflection of the incident light may be suitable. This is the case, for example, when the reflecting particles, irrespective of their shape, allow a reflection on a layer of a metal, such as silver. Such particles may prove relatively neutral in terms of the color of the composition.

Reflecting particles with a metallic or white sheen which can be used in the invention may, for example, reflect light in all the components of the visible range without significantly absorbing one or more wavelengths. The spectral reflectance of these reflecting particles may be, for example, greater than 70% in the 400-700 nm range (the visible range), for example, at least 80%, at least 90%, or at least 95%.

In one embodiment, the light reflected by the reflecting particles may be non-iridescent, for example, in the case of a metallic sheen.

Irrespective of their shape, the reflecting particles may or may not have a multilayer structure. In the case of a multilayer structure, they may have, for example, at least one layer of uniform thickness, for example, of a reflecting material.

When the reflecting particles do not have a multilayer structure, they may comprise, for instance, metal oxides, for example, titanium and iron oxides, which are obtained by synthesis so as to have a substantially planar surface whose state, e.g., non-matt and non-diffusing, allows a specular reflection of the light that is sufficient to produce excessively glossy spots within the cosmetic composition.

When the reflecting particles have a multilayer structure, they may have, for example, a natural or synthetic substrate, for instance, a synthetic substrate that is at least partially coated with at least one layer of at least one reflecting material such as a metal.

Irrespective of the shape of the reflecting particles, the substrate, when synthetic, may be produced with a shape that favors the formation of a reflecting surface after coating, for example, after the deposition of a layer of reflecting material. For example, the substrate may have a planar surface and the layer of reflecting material may have a substantially uniform thickness.

The substrate may comprise at least one material and may be solid or hollow. It may be organic or inorganic. The substrate may be natural or synthetic. In at least one embodiment, the substrate may be chosen from synthetic substrates.

Non-limiting examples of suitable substrates include glasses, ceramics, graphite, metal oxides, aluminas, silicas, silicates, for example, aluminosilicates and borosilicates, and synthetic mica.

In one embodiment, the reflecting material may contain a layer of metal or metal compound.

The layer of metal or metal compound may or may not totally coat the substrate, and the layer of metal or metal compound may be at least partially coated with a layer of another material, e.g., a transparent material. In at least one embodimentm the layer of metal or metal compound may coat the substrate totally, either directly or indirectly, i.e., with the interposition of at least one metallic or non-metallic intermediate layer.

The metal may be chosen, for example, from Ag, Au, Cu, Al, Ni, Sn, Mg, Cr, Mo, Ti, Pt, Va, Rb, W, Zn, Ge, Te, Se, and alloys thereof. In at least one embodiment, the metal may be chosen from Ag, Au, Al, Zn, Ni, Mo, Cr, Cu, and alloys thereof (for example, bronzes and brasses).

The metallic layer may be present in an amount ranging from 0.1 to 50%, for example, from 1 to 20%, of the total weight of the particles, for example, in the case of particles having a substrate coated with silver or gold.

Glass particles coated with a metallic layer may have a dimension ranging, for example, from 10 μm to 300 μm, or from 25 μm to 150 μm. In the case where these particles are in the shape of wafers, the thickness may range, for example, from 0.1 μm to 25 μm, for example, from 0.5 μm to 10 μm, or from 0.5 μm to 5 μm. In the case where these particles are in the shape of spheres, they can have a dimension ranging, for example, from about 10 μm to 100 μm.

Glass particles coated with a metallic layer are described, for example, in Japanese Patent Nos. JP-A-09188830, JP-A-10158450, JP-A-10158541, JP-A-07258460, and JP-A-05017710.

Another non-limiting example of reflecting particles having a mineral substrate coated with a layer of metal is particles having a borosilicate substrate coated with silver, which are also called “white pearlescent products”.

Wafer-shaped particles having a glass substrate coated with silver are sold, for example, under the name MICROGLASS METASHINE REFSX 2025 PS by TOYAL. Particles having a glass substrate coated with nickel/chromium/molybdenum alloy are sold, for example, under the name CRYSTAL STAR GF 550 and GF 2525 by TOYAL.

Irrespective of their shape, the reflecting particles may also be chosen from particles having a synthetic substrate that is at least partially coated with at least one layer of at least one metal compound, for instance, a metal oxide chosen, for example, from titanium oxides, such as TiO₂, iron oxides, such as Fe₂O₃, tin oxides, and chromium oxides, barium sulphate, MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixture or alloys thereof.

Examples of such particles include, but are not limited to, particles having a synthetic mica substrate coated with titanium dioxide and particles of glass coated with brown iron oxide, titanium oxide, and/or tin oxide, such as the particles sold, for example, under the mark REFLECKS® by ENGELHARD.

Other pigments suitable for use in accordance with the present disclosure include those of the METASHINE range, such as the references MC 1120, 1080, 1040, 1020, ME 2040, and MC 2080, marketed by NIPPON SHEET GLASS CO. LTD. These pigments, which are described, for example, in Japanese Patent Application No. JP 2001-11340, are C-GLASS wafers comprising from 65 to 72% of SiO₂ and coated with a layer of titanium oxide of the rutile type (TiO₂). These glass wafers have a mean thickness of 1 micron and a mean size of 80 microns, i.e., a mean size/mean thickness ratio of 80. They exhibit a blue, green, yellow, or silver-tinted sheen according to the thickness of the TiO₂ layer.

Other suitable particles include, but are not limited to, those having a dimension ranging from 80 to 100 μm, having a synthetic mica substrate (fluorophlogopite) coated with titanium dioxide representing 12% of the total weight of the particle, and sold, for example, under the name PROMINENCE by NIHON KOKEN.

The reflecting particles may also be chosen from particles formed of a stack of at least two layers with different refractive indices. These layers may be of a polymeric or metallic nature and may include at least one polymer layer. Thus, the reflecting particles may be particles derived from a multilayer polymer film. Such particles are described, for example, in International Patent Application Publication No. WO 99/36477 and U.S. Pat. Nos. 6,299,979 and 6,387,498.

Non-limiting examples of materials of which the different layers of the multilayer structure can be made include polyethylene naphthalate (PEN) and isomers thereof, for example, 2,6-, 1,4-, 1,5-, 2,7-, and 2,3-PEN; polyalkylene terephthalates, polyimides; polyetherimides; atactic polystyrenes; polycarbonates; polyalkyl methacrylates; polyalkyl acrylates; syndiotactic polystyrene (sPS), syndiotactic poly-alpha-methylstyrenes, syndiotactic polydichlorostyrene, and copolymers and mixtures of these polystyrenes; cellulose derivatives; polyalkylene polymers; fluorinated polymers; chlorinated polymers; polysulphones; polyethersulphones; polyacrylonitriles; poly-amides; silicone resins; epoxy resins; polyvinyl acetate; polyetheramides; ionomeric resins; elastomers; and polyurethanes. Copolymers may also be used, for example, PEN copolymers (for example, copolymers of naphthalene-2,6-, -1,4-, -1,5-, -2,7-, and/or -2,3-dicarboxylic acid or esters thereof with (a) terephthalic acid or esters thereof, (b) isophthalic acid or esters thereof, (c) phthalic acid or esters thereof, (d) alkane glycols, (e) cycloalkane glycols (e.g. cyclohexanedimethanoldiol), (f) alkanedicarboxylic acids, and/or (g) cycloalkanedicarboxylic acids), polyalkylene terephthalate copolymers, and styrene copolymers. In at least one embodiment, each individual layer may include mixtures of two or more of the above polymers or copolymers.

The materials that are intended to make up the different layers of the multilayer structure may be chosen so as to impart the desired reflective appearance to the particles formed.

Reflecting particles comprising a stack of at least two layers of polymers are marketed, for example, by 3M under the name MIRROR GLITTER. These particles contain layers of 2,6-PEN and polymethyl methacrylate in a weight ratio of 80/20. Such particles are described, for example, in U.S. Pat. No. 5,825,643.

As a variant or in addition, the gloss of the reflecting particles may also be due to the reflection of light on a layer of a material of the particle whose refractive index is sufficiently large compared with that of the medium from which the incident light originates.

The cosmetic composition according to the invention may also comprise reflecting particles of different types without going outside the scope of the present disclosure.

Polymer

The oily phase of the compositions according to the present invention further comprises at least one polymer having a weight-average molecular weight of less than 100,000, for example, less than 50,000, comprising (a) a polymer skeleton having repeat hydrocarbon units including at least one heteroatom, and (b) optionally at least one pendant fatty chain and/or at least one terminal fatty chain which may be optionally functionalized, comprise from 6 to 120 carbon atoms and which are bonded to the hydrocarbon repeat units.

As used herein, the term “functionalized chains” is understood to mean alkyl chains comprising at least one functional or reactive group chosen, for example, from amide, hydroxyl, ether groups, oxyalkylene groups, polyoxyalkylene groups, halogen groups, including fluorinated and perfluorinated groups, ester groups, siloxane groups, and polysiloxane groups. Optionally, the hydrogen atoms of at least one fatty chain may be at least partially replaced with fluorine atoms.

According to one embodiment of the present disclosure, these chains may be bonded to the polymer skeleton directly or via an ester group or a perfluorinated group.

As used herein, the term “polymer” is understood to mean a compound comprising at least 2 repeat units, for example, at least 3 repeat units.

As used herein, the term “hydrocarbon repeat units” is understood to mean units comprising from 2 to 80 carbon atoms, for example, from 2 to 60 carbon atoms, comprising hydrogen atoms, and optionally comprising oxygen atoms, it being possible for said units to be linear, branched, or cyclic and saturated or unsaturated. These units also each comprise at least one heteroatom, which may, in at least one embodiment, be non-pendant and situated in the polymer skeleton. The at least one heteroatom may be chosen from nitrogen, sulphur, and phosphorus atoms and groups thereof, and may be optionally associated with at least one oxygen atom. In at least one embodiment, the units may comprise at least one nitrogen atom, for example, a non-pendant nitrogen atom. In another embodiment, these units may further comprise at least one carbonyl group.

In yet another embodiment, the units comprising a heteroatom may be chosen from amide units forming a skeleton of the polyamide type, and carbamate and/or urea units forming a polyurethane, polyurea, and/or polyurea-urethane skeleton. In one embodiment, these units may be chosen from amide units. In another embodiment, the pendant chains may be bonded directly to at least one of the heteroatoms of the polymer skeleton.

This polymer may comprise silicone units or oxyalkylene units between the hydrocarbon units.

Moreover, this polymer of the composition of the present disclosure may comprise from 40 to 98%, for example, from 50 to 95% of fatty chains, based on the total number of units comprising a heteroatom and fatty chains. The nature and proportion of the units comprising a heteroatom depends on the nature of the oily phase and may, in one embodiment, be similar to the polar nature of the oily phase. Thus, the greater the polarity of the units comprising a heteroatom and the higher their proportion in this polymer—which corresponds to the presence of several heteroatoms—the greater is the affinity of this polymer for polar oils. Conversely, the lower the polarity of the units comprising a heteroatom (in one embodiment, they may even be apolar) or the lower their proportion, the greater is the affinity of this polymer for apolar oils.

According to at least one embodiment of the present disclosure, the polymer may be a polyamide. Therefore, further disclosed herein is a composition comprising, in a cosmetically acceptable medium, at least one polyamide polymer having a weight-average molecular weight of less than 100,000, comprising (a) a polymer skeleton comprising amide repeat units, and (b) optionally at least one pendant fatty chain and/or at least one terminal chain which may be optionally functionalized, comprise from 8 to 120 carbon atoms, and are bonded to the amide units.

In one embodiment, the pendant fatty chains may be bonded to at least one of the nitrogen atoms of the amide units of this polymer.

In another embodiment, the fatty chains of this polyamide may represent from 40 to 98%, for example, from 50 to 95% of the total number of amide units and fatty chains.

In a further embodiment, this polymer (for example, this polyamide), may have a weight-average molecular weight of less than 100,000 (e.g., ranging from 1000 to 100,000), for example, less than 50,000 (e.g., ranging from 1000 to 50,000), or ranging from 1000 to 30,000, for example, from 2000 to 20,000, or from 2000 to 10,000.

In yet another embodiment, this polymer (for example, this polyamide), may be insoluble in water, for instance, at 25° C. In still a further embodiment, the polymer does not contain ionic groups.

Non-limiting examples of suitable polymers include polyamides branched by pendant fatty chains and/or terminal fatty chains compring from 6 to 120 carbon atoms, for example, from 8 to 120, or from 12 to 68 carbon atoms, each terminal fatty chain being bonded to the polyamide skeleton by at least one linking group, for instance, an ester group. In one embodiment, these polymers may have a fatty chain at each end of the polymer skeleton, for example, the polyamide skeleton. Other linking groups include, but are not limited to, ether, amine, urea, urethane, thioester, thiourea, and thiourethane groups.

In another embodiment, these polymers may be polymers that result from a polycondensation reaction between a dicarboxylic acid comprising at least 32 carbon atoms (for example, from 32 to 44 carbon atoms) and an amine chosen from diamines comprising at least 2 carbon atoms (for example, from 2 to 36 carbon atoms) and triamines comprising at least 2 carbon atoms (for example, from 2 to 36 carbon atoms). According to one embodiment of the present disclosure, the diacid may be a dimer derived from an ethylenically unsaturated fatty acid comprising at least 16 carbon atoms, for example, from 16 to 24 carbon atoms, such as oleic, linoleic, and linolenic acid. In another embodiment, the diamine may be chosen from ethylenediamine, hexylenediamine, and hexamethylenediamine. A non-limiting example of a suitable triamine is ethylenetriamine. Polymers comprising one or two terminal carboxylic acid groups may be esterified with a monoalcohol comprising at least 4 carbon atoms, for example, from 10 to 36 carbon atoms, from 12 to 24, or from 16 to 24 carbon atoms, for example, 18 carbon atoms.

These polymers include, for example, those described in U.S. Pat. No. 5,783,657 in the name of Union Camp. In one embodiment, these polymers may be polymers of formula (I) below: 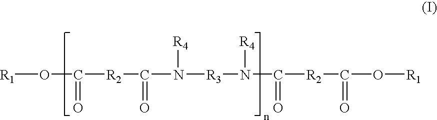
in which
n is a whole number of amide units such that the number of ester groups represents from 10% to 50% of the total number of ester and amide groups;
R₁ is independently chosen from alkyl and alkenyl groups comprising at least 4 carbon atoms, for example, from 4 to 24 carbon atoms;
R₂ is independently chosen from C₄ to C₄₂ hydrocarbon groups, with the proviso that 50% of the groups R₂ are chosen from C₃₀ to C₄₂ hydrocarbon groups;
R₃ is independently chosen from organic groups comprising at least 2 carbon atoms, hydrogen atoms, and optionally at least one entity chosen from oxygen and nitrogen atoms; and
R₄ is independently chosen from hydrogen, C₁ to C₁₀ alkyl groups, and a direct bond to R₃ or to another R₄ such that the nitrogen atom to which both R₃ and R₄ are bonded forms part of a heterocyclic structure defined by R₄—N—R₃, with the proviso that at least 50% of the groups R₄ are hydrogen atoms.

In the case of polymers of formula (I), the optionally functionalized terminal fatty chains are terminal chains bonded to the last heteroatom, in this case nitrogen, of the polyamide skeleton.

For instance, the ester groups of formula (I) which form part of the terminal and/or pendant fatty chains represent from 15 to 40%, for example, from 20 to 35% of the total number of ester and amide groups. In at least one embodiment, n is an integer ranging from 1 to 5, for example, greater than 2. In another embodiment, R₁ may be chosen from C₁₂ to C₂₂ alkyl groups, for example, C₁₆ to C₂₂ alkyl groups. In yet another embodiment, R₂ may be chosen from C₁₀ to C₄₂ (alkylene) hydrocarbon groups. In a further embodiment, at least 50%, for example, at least 75% of the groups R₂ are groups comprising from 30 to 42 carbon atoms. The other groups R₂ may be C₄ to C₁₉, for example, C₄ to C₁₂ hydrogenated groups. In another embodiment, R₃ may be chosen from C₂ to C₃₆ hydrocarbon groups and a polyoxyalkylene groups and R₄ may be hydrogen. In still another embodiment, R₃ may be chosen from C₂ to C₁₂ hydrocarbon groups.

The hydrocarbon groups may be saturated or unsaturated linear, cyclic, or branched groups. Also, the alkyl and alkylene groups may be saturated or unsaturated linear or branched groups.

In at least one embodiment, the polymers of formula (I) may take the form of mixtures of polymers, and these mixtures may comprise a synthetic product corresponding to a compound of formula (I) in which n is 0, i.e., a diester.

Non-limiting examples of polymers suitable for use in the compositions according to the present disclosure include the commercial products sold by Arizona Chemical under the names Uniclear 80 and Uniclear 100. They are sold respectively in the form of an 80% (active substance) gel in a mineral oil and a 100% (active substance) gel. They have a softening point of 88 to 94° C. These commercial products are a mixture of copolymers of a C₃₆ diacid condensed with ethylenediamine, having a weight-average molecular weight of about 6000. The terminal ester groups result from esterification of the residual terminal acid groups with cetyl alcohol, stearyl alcohol, or mixtures thereof (also called cetylstearyl alcohol).

Examples of other polymers suitable for use in the compositions according to the present disclosure include, but are not limited to, polyamide resins resulting from the condensation of an aliphatic dicarboxylic acid and a diamine (including compounds having more than 2 carbonyl groups and 2 amine groups), the carbonyl and amine groups of adjacent individual units being condensed to form an amide linkage. These polyamide resins include, for example, those marketed under the mark Versamid® by General Mills, Inc. and Henkel Corp. (Versamid 930, 744, and 1655) or under the mark Onamid®, for example, Onamid S or C, by Olin Mathieson Chemical Corp. These resins have a weight-average molecular weight ranging from 6000 to 9000. For further information on these polyamides, reference may be made to U.S. Pat. Nos. 3,645,705 and 3,148,125. In at least one embodiment, the polymer may be chosen from Versamid® 930 and Versamid® 744.

Further examples of suitable polyamides include, but are not limited to, those sold by Arizona Chemical under the reference Uni-Rez (2658, 2931, 2970, 2621, 2613, 2624, 2665, 1554, 2623, 2662) and the product sold by Henkel under the reference Macromelt 6212. For further information on these polyamides, reference may be made to U.S. Pat. No. 5,500,209.

Polyamide resins derived from vegetables, such as those described in U.S. Pat. Nos. 5,783,657 and 5,998,570, are also suitable for use in the compositions of the present disclosure.

The polymer present in the composition according to the present disclosure may have a softening point of greater than 65° C., for example, up to 190° C. Its softening point may range, in at least one embodiment, from 70 to 130° C., for example, from 80 to 105° C. In one embodiment, the polymer may be a non-waxy polymer.

By virtue of its optional fatty chain(s), the at least one polymer may have a good solubility in oils, thus producing macroscopically homogeneous compositions, even when the polymer content is high (for example, at least 25%).

This polymer may be present in the composition according to the present disclosure in an amount ranging from 0.01% to 10% by weight, for example, 0.05% to 5% by weight, or from 0.1% to 3% by weight, relative to the total weight of the composition.

In one embodiment, the weight ratio of polymer to silica particles may range from 1:1000 to 1:1, for example, from 1:100 to 1:10, or from 5:1000 to 5:100.

According to at least one embodiment of the present disclosure, the oily phase may comprise an oily phase that is liquid at room temperature, such as those conventionally used in cosmetics. This oily phase may contain polar oils and/or apolar oils.

Polar Oils

Examples of polar oils suitable for use in accordance with the present disclosure include, but are not limited to:
hydrocarbon vegetable oils having a high content of triglycerides comprising fatty acid esters of glycerol in which the fatty acids may have chain lengths ranging from C₄ to C₂₄, it being possible for said chains to be linear or branched and saturated or unsaturated; non-limiting examples of these oils include wheatgerm, maize, sunflower, karite, castor, sweet-almond, macadamia, apricot, soya, cottonseed, alfalfa, poppy, pumpkin, sesame, gourd, colza, avocado, hazelnut, grapeseed, blackcurrant seed, evening primrose, millet, barley, quinoa, olive, rye, safflower, candlenut, passiflora, and muscat rose oils; and caprylic/capric triglycerides such as those sold by Stearineries Dubois or those sold under the names Miglyol 810, 812, and 818 by Dynamit Nobel;
synthetic oils and synthetic esters of the formula RₐCOOR_{b}, in which Rₐ is the residue of a linear or branched fatty acid comprising from 1 to 40 carbon atoms and R_{b} is a hydrocarbon chain, for example, a branched hydrocarbon chain, comprising from 1 to 40 carbon atoms, with the proviso that Rₐ+R_{b}≧10, non-limiting examples include Purcellin oil (cetostearyl octanoate); isononyl isononanoate; the benzoate of a C₁₂ to C₁₅ alcohol; isopropyl myristate; 2-ethylhexyl palmitate; isostearyl isostearate; octanoates, decanoates, and ricinoleates of alcohols or polyalcohols; hydroxylated esters such as isostearyl lactate and diisostearyl malate; and pentaerythritol esters;
synthetic ethers comprising from 10 to 40 carbon atoms;
C₈ to C₂₆ fatty alcohols, such as oleyl alcohol;
C₈ to C₂₆ fatty acids, such as oleic, linolenic, and linoleic acids; and
mixtures thereof.

Apolar Oils

Examples of apolar oils suitable for use in accordance with the present disclosure include, but are not limited to, silicone oils such as volatile or non-volatile linear or cyclic polydimethylsiloxanes (PDMS) that are liquid at room temperature; polydimethylsiloxanes comprising lateral and/or terminal alkyl or alkoxy groups, said groups each comprising from 2 to 24 carbon atoms; phenylated silicones such as phenyltrimethicones, phenyldimethicones, phenyltrimethylsiloxydiphenylsiloxanes, diphenyldimethicones, diphenylmethyldiphenyltrisiloxanes, and 2-phenylethyltrimethylsiloxy silicates; volatile or non-volatile linear or branched hydrocarbons of synthetic or mineral origin, such as volatile paraffin oils (isoparaffins like isododecane) and non-volatile paraffin oils and derivatives thereof, petrolatum, liquid lanolin, polydecenes, hydrogenated polyisobutene, such as Parleam oil, squalane, and arara oil; and mixtures thereof.

In at least one embodiment, the oils may be chosen from apolar oils, for example, a hydrocarbon oil of mineral or synthetic origin, and mixtures of such oils, chosen, for example, from alkanes such as Parleam oil, isoparaffins such as isododecane, squalane, and mixtures thereof. In one embodiment, these oils may be optionally associated with at least one phenylated silicone oil.

In another embodiment, the liquid oily phase may comprise at least one non-volatile oil chosen, for example, from hydrocarbon oils of mineral, vegetable, or synthetic origin, synthetic esters and ethers, silicone oils, and mixtures thereof.

The total liquid oily phase may be present in the composition in an amount ranging from 5 to 99.95%, for example, from 10 to 80%, or from 20 to 75%, relative to total weight of the composition.

Optional Additives

The compositions according to the present disclosure may also comprise at least one agent chosen from pigments, pearlescent products, and/or lakes.

As used herein, the term “pigments” is understood to mean white or colored, mineral or organic, coated or uncoated particles. Examples include, but are not limited to, titanium, zirconium, and cerium dioxides; zinc, iron, and chromium oxides; Prussian blue; chrome hydrate; carbon black; ultramarines (aluminosilicate polysulphides); manganese violet; manganese pyrophosphate; and certain metal powders such as silver and aluminium powders; and mixtures thereof.

As used herein, “pearlescent products” is understood to mean white pearlescent pigments such as mica coated with titanium oxide and bismuth oxychloride, and colored pearlescent pigments such as titanium mica coated with iron oxides, Prussian blue, and chromium oxide, or with an organic pigment of the precipitated type.

Non-limiting examples of lakes which can be used in the compositions of the present disclosure include lakes based on carmine; lakes based on calcium, barium, aluminium, strontium, and zirconium salts; acid colorants; and mixtures thereof.

The at least one agent chosen from pigments, lakes, and pearlescent products may be present in the cosmetic composition according to the present disclosure in an amount ranging from 0.05% to 20% by weight, for example, from 0.1 to 15% by weight, relative to the total weight of the cosmetic composition.

In one embodiment of the present disclosure, the cosmetic composition may further comprise at least one cosmetic active ingredient. Examples of suitable cosmetic active ingredients include, but are not limited to, moisturizers (for example, polyols such as glycerol), vitamins (for example, vitamins C, A, E, F, B, and PP), essential fatty acids, essential oils, ceramides, sphingolipids, liposoluble sun filters, sun filters in the form of nanoparticles, and specific active ingredients for treating the skin (for example, protectants, antibacterials, and anti-wrinkle agents). The at least one active ingredient may be present in the composition in an amount ranging from 0 to 20%, for example, from 0.001 to 15%, relative to the total weight of the composition.

The cosmetic composition may also comprise ingredients commonly used in cosmetics, such as thickeners, surfactants, trace elements, moisturizers, softeners, sequestering agents, perfumes, alkalizing agents, acidifying agents, preservatives, antioxidants, UV filters, and mixtures thereof.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

Procedure

The procedure below was followed for each of the examples:

The pigments were ground in the liquid fatty substances using a triple roll mill.

When the pigments were ground, polybutene was added and the mixture was then agitated by means of a Rayneri agitator while being heated to 100-105° C.

When the mixture was homogeneous, pearlescent products and perfume were added and silica was then introduced gradually, with agitation, the temperature being maintained at 100-105° C. Suspension of the silica took about 20 minutes for 300 g of product. The product obtained was then poured into boilers, either directly while hot or after cooling.

The polymer Uniclear® 100 was then introduced.

### Example 1

A lip make-up product having the following composition was prepared:

| Polybutene | qsp |
|---|---|
| Diisostearyl malate | 9 |
| Pentaerythrityl isostearate | 14 |
| Tridecyl trimellitate | 11 |
| Triglyceride of C₁₈₋₃₆ acid | 20 |
| Bisdiglyceryl polyacyladipate 2 | 18 |
| Uniclear ® 100^{†} | 0.3 |
| Silica | 8 |
| Preservative | 0.51 |
| Pigment, pearlescent products | 6.95 |
| Metashine | 8 |
| Perfume | 0.3 |

^{†}Uniclear ® 100: condensation product of a hydrogenated C₃₆ diacid and ethylenediamine, esterified with stearyl alcohol (weight-average molecular weight: about 4000), marketed by ARIZONA CHEMICAL.

No sedimentation of the particles (pigment, pearlescent products, Metashine) was observed after storage of this product for 3 months at room temperature.

### Example 2

A lip make-up product having the following composition was prepared:

| Polybutene | qsp |
|---|---|
| Diisostearyl malate | 9 |
| Pentaerythrityl isostearate | 14 |
| Tridecyl trimellitate | 11 |
| Triglyceride of C₁₈₋₃₆ acid | 20 |
| Bisdiglyceryl polyacyladipate 2 | 18 |
| Uniclear ® 100 | 0.5 |
| Silica | 8 |
| Preservative | 0.51 |
| Pigment, pearlescent products | 6.95 |
| Metashine | 8 |
| Perfume | 0.3 |

No sedimentation of the particles (pigment, pearlescent products, Metashine) was observed after storage of this product for 3 months at room temperature.

What is claimed is:

1. A cosmetic skin make-up and/or care composition comprising an oily phase comprising suspended silica particles and reflecting particles, wherein the oily phase comprises at least one polymer having a weight-average molecular weight of less than 100,000, comprising
(a) a polymer skeleton comprising hydrocarbon repeat units including at least one heteroatom, and
(b) optionally at least one pendant fatty chain and/or at least one terminal fatty chain which may be optionally functionalized, comprise from 6 to 120 carbon atoms, and which are bonded to the hydrocarbon repeat units.

2. The composition of claim 1, wherein the at least one polymer is chosen from polyamides having a weight-average molecular weight of less than 100,000, comprising
(a) a polymer skeleton comprising hydrocarbon repeat units which are amides, and
(b) optionally at least one pendant fatty chain and/or at least one terminal fatty chain which may be optionally functionalized, comprise from 8 to 120 carbon atoms, and which are bonded to the hydrocarbon repeat units which are amides.

3. The composition of claim 2, wherein the fatty chains represent from 40 to 98% of the total number of amide units and fatty chains.

4. The composition of claim 2, wherein the fatty chains represent from 50 to 95% of the total number of amide units and fatty chains.

5. The composition of claim 2, wherein the pendant fatty chains are bonded directly to at least one of the nitrogen atoms of the amide units.

6. The composition of claim 1, wherein the average molecular weight of the polymer ranges from 1000 to 100,000.

7. The composition of claim 6, wherein the average molecular weight of the polymer ranges from 1000 to 50,000.

8. The composition of claim 7, wherein the average molecular weight of the polymer ranges from 1000 to 30,000.

9. The composition of claim 1, wherein the weight-average molecular weight of the polymer ranges from 2000 to 20,000.

10. The composition of claim 9, wherein the weight-average molecular weight of the polymer ranges from 2000 to 10,000.

11. The composition of claim 1, wherein the at least one terminal fatty chain is bonded to the skeleton by linking groups.

12. The composition of claim 11, wherein the linking groups are ester groups.

13. The composition of claim 1, wherein the at least one fatty chain comprises from 12 to 68 carbon atoms.

14. The composition of claim 1, wherein the polymer is chosen from polyamides of formula (I): 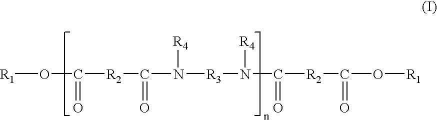
in which
n denotes a whole number of amide units such that the number of ester groups represents from 10% to 50% of the total number of ester and amide groups;
R₁ is independently chosen from alkyl and alkenyl groups comprising at least 4 carbon atoms;
R₂ is independently chosen from C₄ to C₄₂ hydrocarbon groups, with the proviso that 50% of the groups R₂ are chosen from C₃₀ to C₄₂ hydrocarbon groups;
R₃ is independently chosen from organic groups comprising at least 2 carbon atoms, hydrogen, and optionally at least one entity chosen from oxygen and nitrogen; and
R₄ is independently chosen from hydrogen, C₁ to C₁₀ alkyl groups, and a direct bond to R₃ or to another R₄ such that the nitrogen atom to which both R₃ and R₄ are bonded forms part of a heterocyclic structure defined by R₄—N—R₃, with the proviso that at least 50% of the groups R₄ are hydrogen.

15. The composition according to claim 14, wherein R₁ is independently chosen from alkyl and alkenyl groups comprising from 4 to 24 carbon atoms.

16. The composition of claim 14, wherein R₁ is chosen from C₁₂ to C₂₂ alkyl groups.

17. The composition of claim 14, wherein R₂ comprises from 30 to 42 carbon atoms.

18. The composition of claim 1, wherein the at least one polymer is present in the composition in an amount ranging from 0.01% to 10% by weight, relative to the total weight of the composition.

19. The composition of claim 18, wherein the at least one polymer is present in the composition in an amount ranging from 0.05% to 5% by weight, relative to the total weight of the composition.

20. The composition of claim 19, wherein the at least one polymer is present in the composition in an amount ranging from 0.1% to 3% by weight, relative to the total weight of the composition.

21. The composition of claim 1, wherein the silica particles are present in the composition in an amount ranging from 0.1% to 12% by weight, relative to the total weight of the composition.

22. The composition of claim 21, wherein the silica particles are present in the composition in an amount ranging from 0.5% to 10% by weight, relative to the total weight of the composition.

23. The composition of claim 22, wherein the silica particles are present in the composition in an amount ranging from 6% to 8% by weight, relative to the total weight of the composition.

24. The composition of claim 1, wherein the reflecting particles have a spectral reflectance in the visible spectrum of at least 70%.

25. The composition of claim 1, wherein the reflecting particles have a dimension of less than or equal to 250 μm.

26. The composition of claim 25, wherein the reflecting particles have a dimension of less than or equal to 150 μm.

27. The composition of claim 26, wherein the reflecting particles have a dimension of less than or equal to 100 μm.

28. The composition of claim 1, wherein the reflecting particles have a dimension of at least 10 μm.

29. The composition of claim 28, wherein the reflecting particles have a dimension ranging from 20 to 80 μm.

30. The composition of claim 1, wherein the reflecting particles are present in the composition in an amount ranging from 0.1 to 20%, relative to the total weight of the composition.

31. The composition of claim 30, wherein the reflecting particles are present in the composition in an amount ranging from 1 to 15%, relative to the total weight of the composition.

32. The composition of claim 31, wherein the reflecting particles are present in the composition in an amount ranging from 1 to 10%, relative to the total weight of the composition.

33. The composition of claim 1, wherein the reflecting particles are in the shape of wafers or spheres.

34. The composition of claim 1, wherein the reflecting particles comprise particles having a natural or synthetic substrate that is at least partially coated with a layer of at least one metal.

35. The composition of claim 34, wherein the at least one metal is chosen from Ag, Au, Cu, Al, Zn, Ni, Mo, Cr, and mixtures and alloys thereof.

36. The composition of claim 36, wherein the at least one metal is chosen from Ag and its alloys.

37. The composition of claim 34, wherein the substrate is chosen from substrates comprising at least one material, organic substrates, inorganic substrates, glasses, ceramics, metal oxides, aluminas, silicas, silicates, synthetic mica, and mixtures thereof.

38. The composition of claim 37, wherein the silicates are chosen from aluminosilicates and borosilicates.

39. The composition of claim 1, wherein the reflecting particles are at least partially composed of particles having a synthetic substrate that is at least partially coated with at least one layer of at least one metal compound.

40. The composition of claim 39, wherein the at least one metal compound is chosen from metal oxides.

41. The composition of claim 39, wherein the synthetic substrate is chosen from substrates comprising at least one materials, organic substrates, inorganic substrates, glasses, ceramics, metal oxides, aluminas, silicas, silicates, synthetic mica, and mixtures thereof.

42. The composition of claim 39, wherein the metal compound is chosen from titanium oxides, iron oxides, tin oxides, barium sulphate, MgF₂, CeF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures thereof.

43. The composition of claim 42, wherein the metal oxide is chosen from TiO₂ and Fe₂O₃.

44. The composition of claim 42, wherein the metal compound is chosen from titanium oxides, iron oxides, tin oxides, and mixtures thereof.

45. The composition of claim 44, wherein the metal compound is TiO₂.

46. The composition of claim 1, wherein the reflecting particles comprise particles formed of a stack of at least two layers with different refractive indices.

47. The composition of claim 46, wherein the reflecting particles comprise particles formed of a stack of at least two layers of polymers.

48. The composition of claim 1, wherein the reflecting particles are at least partially composed of particles of at least one metal oxide.

49. The composition of claim 48, wherein the at least one metal oxide is chosen from iron oxides and titanium oxides.

50. The composition of claim 1, wherein the reflecting particles are present in the composition in an amount ranging from 0.01% to 10% by weight, relative to the total weight of the composition.

51. The composition of claim 1, further comprising at least one agent chosen from pigments, pearlescent products, and/or lakes.

52. The composition of claim 1, further comprising at least one active ingredient chosen from moisturizers, vitamins, essential fatty acids, essential oils, ceramides, sphingolipids, liposoluble sun filters, and sun filters in the form of nanoparticles.

53. The composition of claim 1, further comprising at least one ingredient chosen from thickeners, surfactants, trace elements, moisturizers, softeners, sequestering agents, perfumes, alkalizing agents, acidifying agents, preservatives, antioxidants, UV filters, and mixtures thereof.

54. The composition of claim 1, wherein the weight ratio of polymer to silica particles ranges from 1:1000 to 1:1.

55. The composition of claim 54, wherein the weight ratio of polymer to silica particles ranges from 1:100 to 1:10.

56. The composition of claim 55, wherein the weight ratio of polymer to silica particles ranges from 5:1000 to 5:100.

57. A process for the preparation of a cosmetic skin make-up and/or care composition comprising mixing silica particles, reflecting particles, and at least one polymer having a weight-average molecular weight of less than 100,000,
wherein the at least one polymer comprises:
(a) a polymer skeleton comprising hydrocarbon repeat units including at least one heteroatom, and
(b) optionally at least one pendant fatty chain and/or at least one terminal fatty chain which may be optionally functionalized, comprise from 6 to 120 carbon atoms, and which are bonded to the hydrocarbon repeat units.

58. A gloss comprising a cosmetic composition comprising an oily phase comprising suspended silica particles and reflecting particles, wherein the oily phase comprises at least one polymer having a weight-average molecular weight of less than 100,000, comprising
(a) a polymer skeleton comprising hydrocarbon repeat units including at least one heteroatom, and
(b) optionally at least one pendant fatty chain and/or at least one terminal fatty chain which may be optionally functionalized, comprise from 6 to 120 carbon atoms, and which are bonded to the hydrocarbon repeat units.

59. A method for obtaining a glossy deposit comprising applying a cosmetic composition to a substrate, wherein the cosmetic composition comprises an oily phase comprising suspended silica particles and reflecting particles, wherein the oily phase comprises at least one polymer having a weight-average molecular weight of less than 100,000, comprising
(a) a polymer skeleton comprising hydrocarbon repeat units including at least one heteroatom, and
(b) optionally at least one pendant fatty chain and/or at least one terminal fatty chain which may be optionally functionalized, comprise from 6 to 120 carbon atoms, and which are bonded to the hydrocarbon repeat units.
